(19) United States
(12) Patent Application Publication
(10) Pub. No.: US 2010/0317925 A1
(43) Pub. Date: Dec. 16, 2010

(54) SUCTION-ASSISTED TISSUE STABILIZERS

(76) Inventors: Michael J. Banchieri, Discovery Bay, CA (US); Dwight P. Morejohn, Davis, CA (US); Tamer Ibrahim, Pleasant Hill, CA (US)

Correspondence Address: HENRICKS SLAVIN AND HOLMES LLP;SUITE 200, 840 APOLLO STREET, EL SEGUNDO, CA, 90245, US

(21) Appl. No.: 12/483,863

(22) Filed: Jun. 12, 2009

(51) Int. Cl. A61B 1/32

(52) U.S. Cl. 600/210

(57) ABSTRACT

Suction assisted tissue stabilizers that include portions that deflect when force is applied thereto and return to their initial shape when the force is removed.

### BACKGROUND

1. Field

The present inventions relate generally to suction-assisted tissue stabilizers.

2. Description of the Related Art

Suction-assisted tissue stabilizers (“tissue stabilizers”) are used in surgical procedures to stabilize, position, and/or inhibit the physiological movement of tissue. Some tissue stabilizers include soft suction members that are carried on two or more rigid supports. The rigid supports are, in turn, carried on an articulating arm. The rigid supports in some tissue stabilizers are connected to a mechanical linkage that drives the rigid supports away from one another when the orientation of the articulating arm is fixed by applying tension to the articulating arm's tensioning cable. The target tissue structure may be secured to the tissue stabilizer by applying negative pressure to the suction members prior to driving rigid supports away from one another. The tissue structure will be pulled into tension, which reduces the difficulty of the surgical procedure being performed on the tissue.

The present inventors have determined that conventional tissue stabilizers are susceptible to improvement. For example, the present inventors have determined that conventional tissue stabilizers which apply tension force to the tissue are unnecessarily complex and are difficult to use.

### SUMMARY

A tissue stabilizer in accordance with one implementation of a present invention includes a frame having a resilient portion and at least one suction member having at least one suction port carried by the frame. Surgical systems in accordance with various implementations of at least some of the present inventions includes an arm and a tissue stabilizer, associated with the arm, that has a frame with a resilient portion and at least one suction member having at least one suction port carried by the frame.

A tissue stabilizer in accordance with one implementation of a present invention includes first and second suction zones, defines an initial shape, and is configured such that at least one of the first and second suction zones will move a distance at least 1 mm toward or away from one another in response to the application of a force of at least 1 pound thereto and the tissue stabilizer will return to the initial shape when the force is removed. Surgical systems in accordance with various implementations of at least some of the present inventions includes an arm and a tissue stabilizer that has first and second suction zones, defines an initial shape, and is configured such that at least one of the first and second suction zones will move a distance at least 1 mm toward or away from one another in response to the application of a force of at least 1 pound thereto and the tissue stabilizer will return to the initial shape when the force is removed.

The above described and many other features of the present inventions will become apparent as the inventions become better understood by reference to the following detailed description when considered in conjunction with the accompanying drawings.

### BRIEF DESCRIPTION OF THE DRAWINGS

Detailed descriptions of exemplary embodiments will be made with reference to the accompanying drawings.

FIG. 1 is a perspective view of a surgical system in accordance with one embodiment of a present invention.

FIG. 2 is a bottom view of a tissue stabilizer and a suction tube in accordance with one embodiment of a present invention.

FIG. 3 is a front view of the tissue stabilizer illustrated in FIG. 2.

FIG. 4 is a top view of a portion of the tissue stabilizer illustrated in FIG. 2.

FIG. 5 is a bottom view of a portion of the tissue stabilizer illustrated in FIG. 2.

FIG. 5A is a perspective view of a portion of the tissue stabilizer illustrated in FIG. 2.

FIG. 6 is an enlarged view of the tissue stabilizer illustrated in FIG. 2.

FIG. 6A is section view taken along line 6A-6A in FIG. 6.

FIG. 7 is a perspective view of a portion of the tissue stabilizer illustrated in FIG. 2.

FIGS. 8A-8E are front view, partial section views showing a method of using the tissue stabilizer illustrated in FIG. 2.

FIGS. 9A-9E are top views showing a method of using the tissue stabilizer illustrated in FIG. 2.

FIG. 10 is a section view of a linkage assembly in accordance with one embodiment of a present invention.

FIG. 11 is a section view of a portion of a linkage assembly in accordance with one embodiment of a present invention.

FIG. 12 is a section view of a portion of a linkage assembly in accordance with one embodiment of a present invention.

FIGS. 13A and 13B are section views of links in accordance with one embodiment of a present invention.

FIGS. 13C and 13D are section views of links in accordance with one embodiment of a present invention.

FIGS. 14A and 14B are section views of links in accordance with one embodiment of a present invention.

FIGS. 14C and 14D are section views of links in accordance with one embodiment of a present invention.

FIGS. 14E and 14F are section views of links in accordance with one embodiment of a present invention.

FIG. 15 perspective view of a portion of a cable in accordance with one embodiment of a present invention.

FIG. 16A is a plan view of a connector collar in accordance with one embodiment of a present invention.

FIG. 16B is another plan view of the connector collar illustrated in FIG. 16A.

FIG. 16C is a perspective view of the connector collar illustrated in FIG. 16A.

FIG. 17A is a section view of a connector inner cylinder in accordance with one embodiment of a present invention.

FIG. 17B is a plan view of the connector inner cylinder illustrated in FIG. 17A.

FIG. 17C is a perspective view of the connector inner cylinder illustrated in FIG. 17A.

### DETAILED DESCRIPTION OF THE EXEMPLARY EMBODIMENTS

The following is a detailed description of the best presently known modes of carrying out the inventions. This description is not to be taken in a limiting sense, but is made merely for the purpose of illustrating the general principles of the inventions.

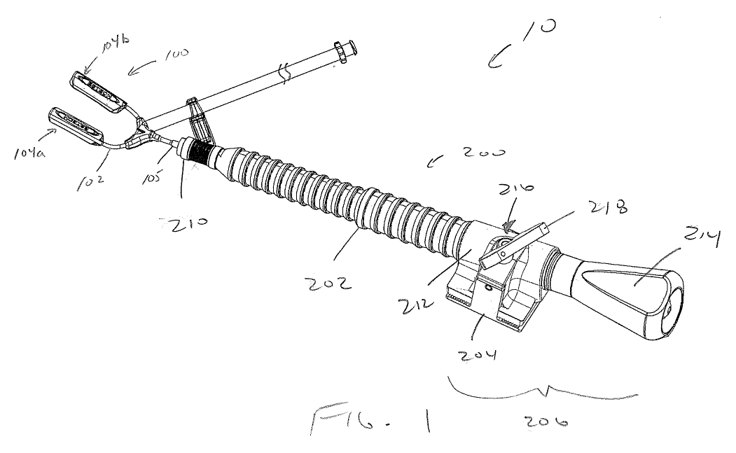

An exemplary surgical system in accordance with one embodiment of a present invention is generally represented by reference numeral 10 in FIG. 1. The surgical system includes a tissue stabilizer apparatus 100 carried on a flexible articulating arm (or “arm”) 200. Exemplary tissue stabilizer apparatus, such as tissue stabilizer apparatus 100, which may be releasably or permanently coupled to the arm 200, are discussed in greater detail below with reference to FIGS. 1-9C. The exemplary arm 200 is discussed in greater detail below with reference to FIGS. 1 and 10-17C.

As illustrated for example in FIGS. 2-3, the exemplary tissue stabilizer apparatus 100 consists of a resilient suction-assisted tissue stabilizer 102, with a pair of suction zones 104a and 104b, and a connector 105 that may be used to releasaby connect the tissue stabilizer to, for example, the flexible articulating arm 200. The tissue stabilizer 102 includes a pair of suction members 106 that are carried on a frame 108, and the suction zones 104a and 104b are each defined by one of the suction members and a portion of the frame. Each suction member 106 has one or more suction ports 110. There are four (4) equally sized, generally circular suction ports 110 on each suction member 106 in the exemplary implementation. In other implementations, the suction ports may vary in size within the same suction member and/or may be shaped other than circular in shape, e.g. elliptical, trapezoidal or square in shape. The frame 108 in the in the illustrated implementation is generally U-shaped and has curved portion 112 and a pair of straight portions 114. Other suitable shapes include, but are not limited to, oval, V-shape and starfish-like shapes. In the illustrated implementation, the curved portion 112 is angled relative to a plane defined by the pair of straight portions 114 by an angle of, for example, 200 (FIG. 5A). The tissue stabilizer 102 also has a frame port 116 that may be connected to a negative pressure source (not shown) by a tube 118 alone, or by the tube 118 in combination with other suitable tubes and structures. The tube 118 may be provided with a clip 119 that may be used to secure a portion of the tube to the arm 200 and prevent the tube from interfering with the surgical procedure. In other implementations, a similar tube may be located within the arm.

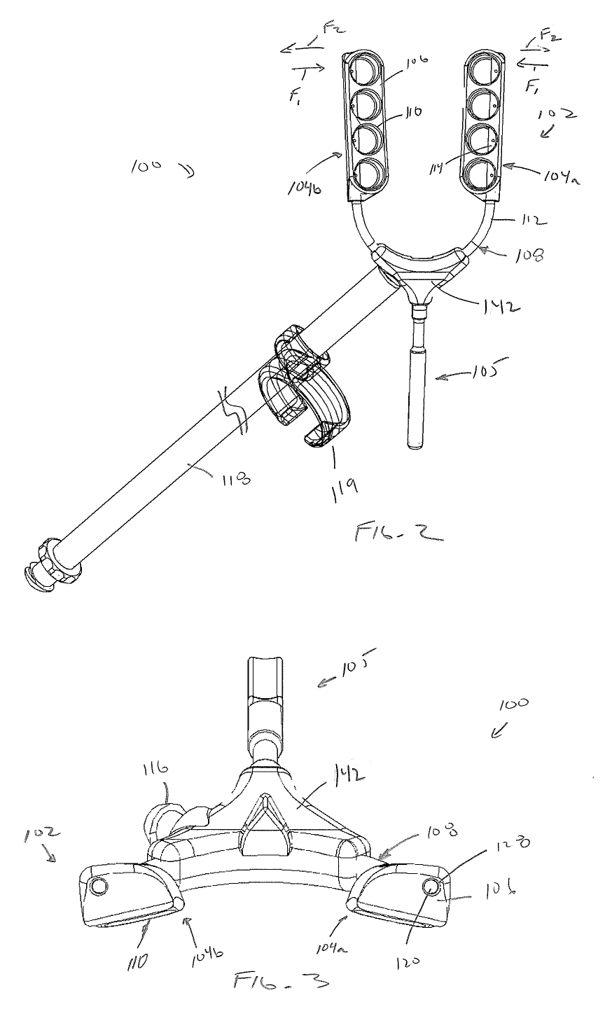

There are variety of ways to establish a fluidic path from the frame port 116 to the suction ports 110. Referring first to FIGS. 4 and 5, the exemplary frame 108 is a hollow (or “tubular”) structure which has an aperture 119 (FIG. 5A) that is aligned with the frame port 116, closed distal ends 120 and a plurality of apertures 122. The apertures 122 are located within, or are exposed to, the suction ports 110. So configured, the frame 108 forms part of the fluid path that connects the suction ports 110 to the source of negative pressure. The configuration of the exemplary suction members 106 also, among other things, establishes a fluidic connection between the frame apertures 122 and the suction ports 110. To that end, and referring to FIGS. 3 and 6-7, the exemplary suction members 106 each include a main body 124 with a tissue engagement surface 126 and a frame lumen 128 for one of the frame straight portions 114. The suction ports 110, which have a side wall 130 and an end wall 132, terminate at the tissue engagement surface 126 and have an opening 134 into the frame lumen 128. The openings 134 result in segments of the associated frame straight portion 114, i.e. those segments that include an aperture 122, being located within or exposed to the suction ports 110. The location of the frame lumen 128 is such that the apertures 122 are off-center relative to the suction ports 106 in the illustrated embodiment, which reduces the likelihood that an upwelling of tissue into the suction ports will obstruct the apertures.

It should also be noted that, in other implementations, where the frame may be either hollow or solid, external tubes may be carried on the exterior of the frame and connected to the suction ports 110. Also, in those instances where the frame is in the form of a hollow tube, the tube need not be circular in cross-section as it is in the illustrated embodiment.

Referring again to FIGS. 4 and 5, the stabilizer frame 108 in the exemplary tissue stabilizer 102 includes a curved frame plate 138 that is mounted on the curved portion 112. The connector 105 is mounted onto the frame 108 by a y-shaped member 140 that is secured to the curved frame plate 138. The curved frame plate 138 also supports the frame port 116, includes an aperture (not shown) that is aligned with the frame port as well as with the corresponding aperture 119 in the frame 108, and forms an air tight seal around the frame port. Turning to FIG. 6, part of frame curved portion 112, as well as the curved plate 138 and the y-shaped member 140, may be covered by a smooth atraumatic structure 142. The atraumatic structure 142, which may be formed from a relatively rigid material such as polycarbonate, prevents sharp edges from damaging tissue. The atraumatic structure 142 is also substantially stiffer than the stabilizer frame 108 and, accordingly, the lateral ends 144a and 144b of the atraumatic structure create fulcrums (or “pivot points”) about which the suction regions 104a and 104b (and frame 108) deflect when the forces described below are applied to the suction regions.

As alluded to above, the tissue stabilizer is also resilient. As used herein, a “resilient” structure is a structure that can be deflected more than an insubstantial distance by pushing or pulling at least one portion of the structure relative to another portion by hand, e.g. at least about 1 mm of deflection when applying a level of force that can be applied by the human thumb and forefinger, and will return (or “spring back”) to its original (or “unstressed”) state (or “shape” or “orientation”) when released. Put another way, the resilient structure can be elastically deformed by hand a distance suitable for the associated surgical procedure without exceeding the elastic limit (which would result in plastic deformation) and will return to original state when the deformation force is removed. The spring constant of the tissue stabilizer 102 may be, in some implementations, about 0.3 pounds force/1 mm defection.

In the exemplary context of the illustrated embodiment, the materials and configuration (discussed below) of the tissue stabilizer 102 are such that the suction zones 104a and 104b can be deflected towards one another to a point where the distance therebetween has been reduced by about 25% to about 100% (i.e. the distal portions of the suction members 106 contact one another) when forces F1 (FIG. 2) of about 2 pounds force to 4 pounds force are applied to distal ends of the stabilizer 102 by, for example, the thumb on one suction zone and the forefinger of the same hand on the other. The suction zones 104a and 104b will spring back to their initial orientation, thereby returning the stabilizer 102 to its initial shape, when the forces are removed. Similarly, the suction zones 104a and 104b can be deflected away from one another by the same distance when opposite forces F2 of the magnitude described above are applied to distal ends of the suction zones, and the suction zones will spring back to their initial orientation when the forces are removed. The exemplary tissue stabilizer 102 may also be deflected out of plane. For example, and referring to the orientation illustrated in FIG. 3, one of the suction zones 104a and 104b may be deflected upwardly and the other deflected downwardly by applying the forces described above, and the suction zones will spring back to the state illustrated in FIG. 3 when the forces are removed.

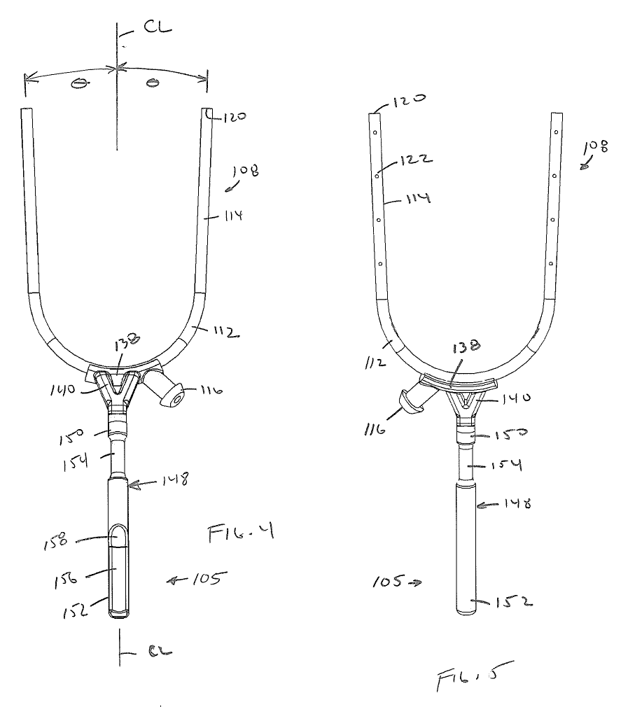

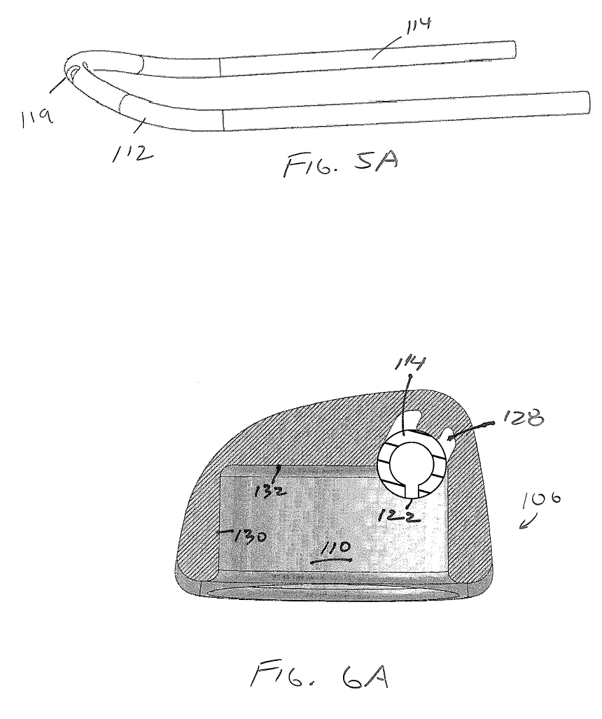

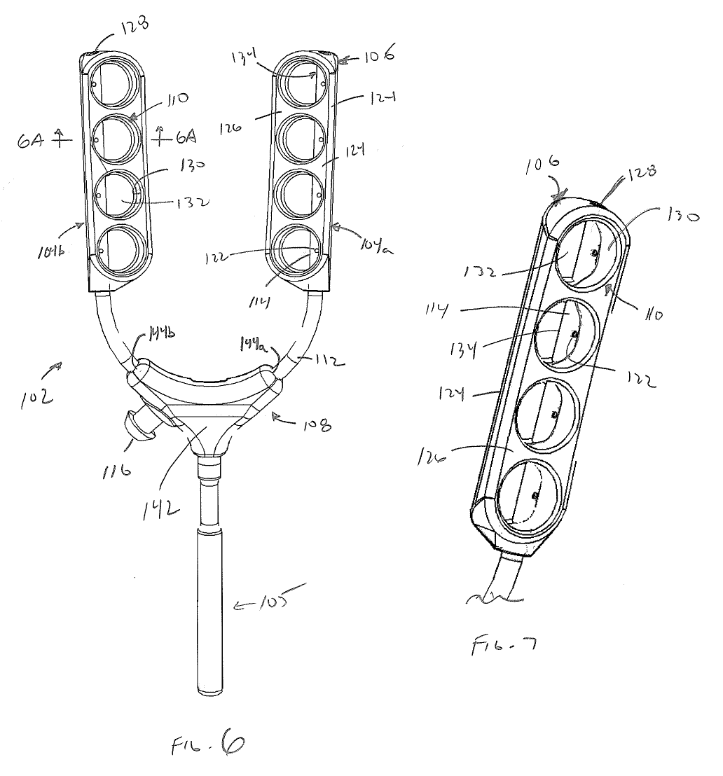

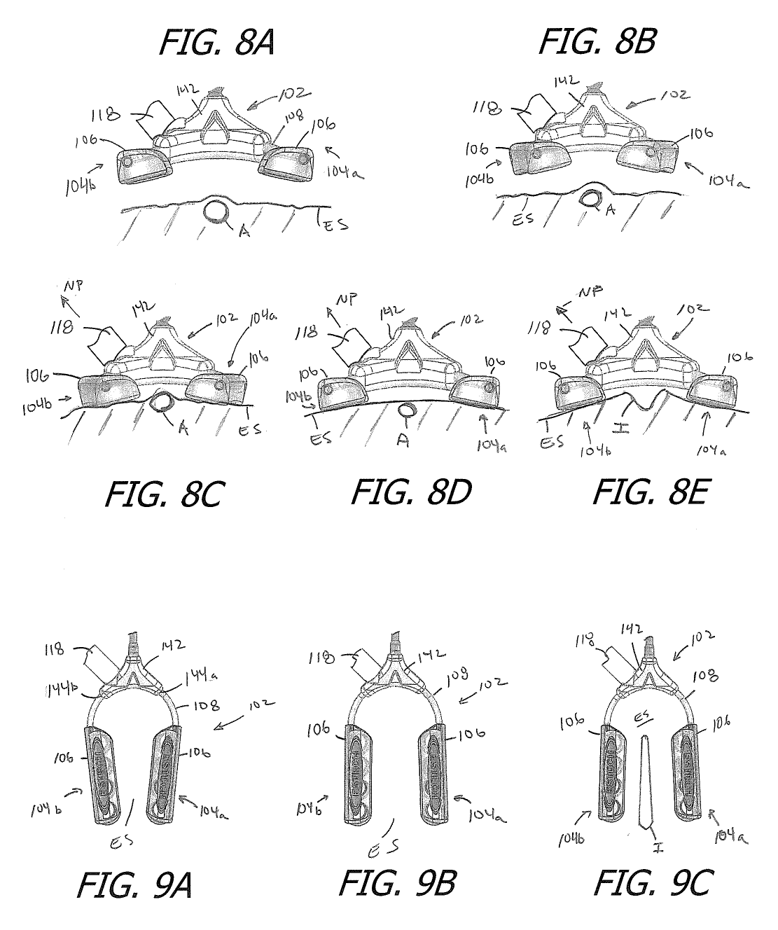

There are a number of advantages associated with the resilient nature of the exemplary tissue stabilizer 102. For example, the tissue stabilizer 102 may be used to spread a tissue structure during a surgical procedure by simply pressing the suction zones 104a and 104b together, positioning the suction zones on the target tissue surfaces, connecting the suction ports 110 to a source of negative pressure to secure the tissue stabilizer to tissue, and releasing the suction zones 104a and 104b. Depending on the tissue structure and the manner in which the tissue stabilizer is applied, the tissue stabilizer 102 will return all of the way, or part of the way, back to its initial orientation. In those instances where the return is partial because the tissue structure prevents full return, the remainder of the return may occur when an incision is made in the tissue structure between the suction zones 104a and 104b and the resiliency of the tissue stabilizer 102 spreads the tissue on opposite sides of the incision. This aspect of at least some of the present inventions is discussed below with reference to FIGS. 8A-9C. Alternatively, a tissue structure may be compressed by forcing the suction zones 104a and 104b away from one another prior to securing the tissue stabilizer 102 to tissue. The tissue stabilizer 102 is also relatively simple and lacks the mechanical linkage associated with conventional tissue stabilizers capable of tensioning tissue.

Although the present tissue stabilizers are not so limited, tissue stabilizers may be configured such that the suction zones are not parallel to one another to one another when the tissue stabilizer is in its unstressed state. The exemplary tissue stabilizer 102 is configured such that the first and second suction zones 104a and 104b are angled away from one another, i.e. the distance between the proximal ends of the suction zones is greater than the distance between the distal ends of the suction zones. More specifically, and referring to FIG. 4, the frame straight portions 114 may be angled relatively to the stabilizer centerline CL by an angle θ of about 10 to about 80 and, in the illustrated embodiment, the angle is about 2.50. There is a similar angular relationship between the suction members 106 that are carried by the frame straight portions 114. The first and second suction zones 104a and 104b may, however, be applied to the tissue in such a manner that the suction zones will be parallel to one another, and the tissue held under tension, prior to an incision. The tissue stabilizer 102 will also spread the tissue as an incision is made and the remaining stress in the frame 108 returns the suction zones 104a and 104b to their original orientation.

To that end, and referring to FIGS. 8A-9C, in one exemplary method, the tissue stabilizer 102 may be employed in a surgical procedure where an is to be formed in the epicardial surface ES parallel to an artery A. First, the tissue stabilizer 102 may be positioned above the epicardial surface ES with the suction zones 104a and 104b in their unstressed state (FIG. 8A). The suction zones 104a and 104b may the be deflected inwardly by pressing the distal ends of the suction members 106 and frame 108 inwardly, i.e. by applying compressive force (FIGS. 8B and 9A). The deflected tissue stabilizer 102, while still under compressive force, may be placed on the epicardial surface ES and connected to a source of negative pressure, thereby securing the suction zones 104a and 104b to the epicardial surface (FIG. 8C). The compressive force may then be removed, and the tissue stabilizer 102 will apply tension force to the epicardial surface ES. More specifically, the stored energy will drive the frame 108 toward the original orientation until the tissue itself prevents further movement (FIGS. 8D and 9B). Here, the suction zones 104a and 104b (as well as the suction members 106 and frame straight portions 114) are parallel to one another. When an incision I is made in the tissue, the tissue will no longer be able to prevent the tissue stabilizer 102 from returning to its unstressed state and, as the suction zones 104a and 104b move apart, the tissue stabilizer will spread the incision (FIGS. 8E and 9C).

A variety of materials and configurations may be employed in a manner that results in a resilient tissue stabilizer that functions in the manner described above. In the illustrated embodiment, the U-shaped tissue stabilizer frame 108 is a tubular structure formed from stainless steel (which has been hardened by cold working to make it resilient) with an outer diameter of about 1.8 mm and a wall thickness of about 0.28 mm. The length of the frame curved portion 112 is about 40 mm, while the length of the straight portions 114 is about 30 mm. The distance between the straight portions 114 is about 27 mm at the proximal end and about 29 mm at the distal end. Other suitable materials include, but are not limited to, metals such as spring steel, nitinol and titanium and plastics such as polyurethane that will also exhibit elastic deformation over the intended range of motion. Suitable materials for the suction members 106 include, but are not limited to, soft, low-durometer materials such as silicone rubber or polyurethane. The length of the suction members 106 in the illustrated embodiment is about the same as the length of the frame straight portions, i.e. about 30 mm, and the width is about 8 mm. The diameter of the suction ports 110 is about 6 mm. The present tissue stabilizers may be manufactured by any suitable process. For example, the suction members 106 and frame 108 may be separately formed and assembled, or the suction members may be molded onto the frame.

As noted above, the present tissue stabilizers are not limited to those with U-shaped frame. By way of example, but not limitation, resilient stabilizers in accordance with the present inventions may be configured with more than two suction zones, such as a starfish-like shape. Other resilient stabilizers may include one or more suction zones that are associated with a rigid portion (i.e. not deflectable by hand under normal surgical conditions) and one or more suction zones that can be deflected in the manner described above.

It should also be noted that the above-described resilient displacement and return of suction regions may be provided in ways that do not rely on the resiliency of the frame materials. For example, and in the context of the exemplary tissue stabilizer 102, the frame may, in other implementations, be formed from rigid material and have portions that are pivotably connected to the lateral ends 144a and 144b of the rigid atraumatic structure 142 by suitable joints. One or more springs may be used to hold the pivotable frame portions in their original state prior to the application of forced thereto, and to return the pivotable frame portions to their original state when the force is removed, in a manner similar to that described above with reference to FIGS. 8A-9C. In another implementation, a rigid fame may have a scissors-like configuration with a spring on one side of the pivot point and the suction zones on the other.

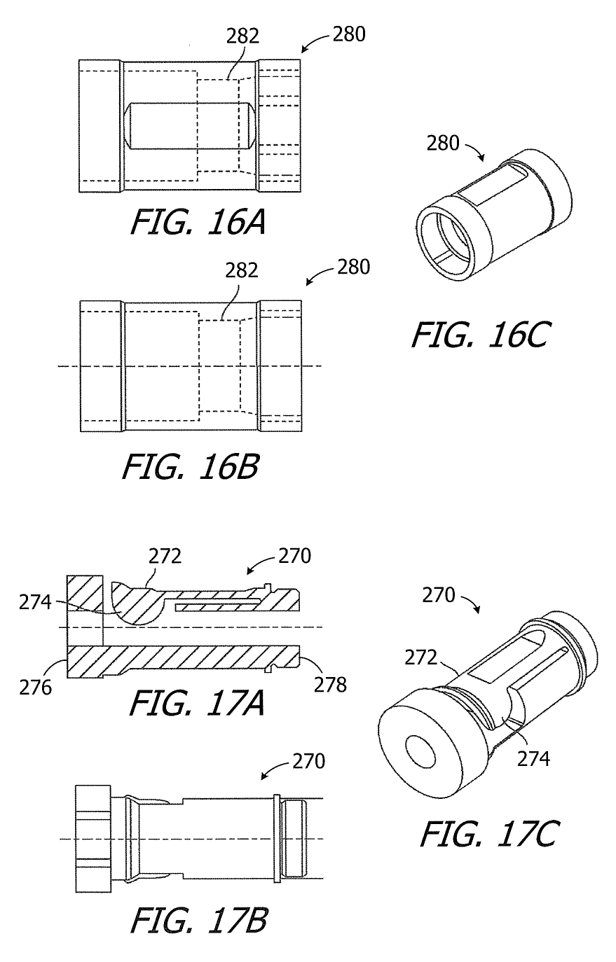

The connector that releasably secures the tissue stabilizer apparatus 100 to the associated flexible articulating arm 200 may be any connector that is suitable for use with the corresponding connector 210 (discussed below) on the articulating arm. In the illustrated embodiments, the connector 105 includes a shaft 148 with first and second end portions 150 and 152 connected to one another by an intermediate portion 154. The outer diameter of the intermediate portion 154 is less than that of the end portions 150 and 152 to enable the user to angle the tissue stabilizer relative to the connector 210 while maintaining a stable connection to the articulating arm 200. The second end portion 152 includes a channel 156 and a spherical indentation 158 that cooperate with the connector 210 in the manner described below with reference to FIGS. 16A-17C to allow the tissue stabilizer apparatus 100 to be easily secured to, and removed from, the articulating arm 200 by hand during the course of normal use.

The connector 105 is but one example of a structure which performs the function releasably securing a tissue stabilizer to a corresponding connector on an arm, such as a flexible articulating arm or some other type of arm. Other exemplary structures which perform the function of releasably securing a tissue stabilizer to an arm include, but are not limited to, the following. A quick-connect, which is configured to be releasably connected to a corresponding structure (e.g. a cylindrical shaft) on the arm, may be provided on the tissue stabilizer apparatus. Alternatively, the arm may be provided with the quick-connect and the tissue stabilizer apparatus may be provided with a corresponding structure (e.g. a cylindrical shaft). In either case, the quick-connect may be configured such that the quick-connect collar slides distally or proximally to engage the post. The tissue stabilizer apparatus may be provided with a male (or female) threaded connector and the arm may be provided with a corresponding female (or male) threaded connector. The tissue stabilizer apparatus and/or the arm may be provided with a magnetic connector. The tissue stabilizer apparatus may be provided with a ball that is configured to be received by a collet on the arm, or the arm may be provided with a ball that is configured to be received by a collet on the tissue stabilizer apparatus. In either case, a cable or a rod may be used to retract the collet into the collar. The arm (or tissue stabilizer apparatus) may be provided with a hollow cylinder and set screw arrangement and the tissue stabilizer apparatus (or arm) may be provided with a shaft that is received within the cylinder. The arm (or tissue stabilizer apparatus) may be provided with a hollow cylinder that has one or more internal indentations and the tissue stabilizer apparatus (or arm) may be provided with a shaft that has one or more outwardly biased depressible members that fit into the indentations. The arm (or tissue stabilizer apparatus) may be provided with a chuck and the tissue stabilizer apparatus (or arm) may be provided with a shaft that is received within the chuck. The tissue stabilizer apparatus (or arm) may be provided with a shaft including one or more transverse notches and the arm (or tissue stabilizer apparatus) may be provided with a hollow cylinder that has one or more transverse holes. After the shaft is inserted into the hollow cylinder such that the notches are aligned with the holes, pins may be placed in the holes to prevent the shaft from moving.

The tissue stabilizer described above may, in other implementations, be a permanent part of a surgical system such as, for example, surgical systems that include a flexible articulating arm. Here, the tissue stabilizer will be permanently connected to the arm through the use of instrumentalities, such as adhesive, weld(s), and/or screws or other mechanical fasteners, that do not allow the tissue stabilizer to be removed without disassembly or destruction of at least that portion of the system.

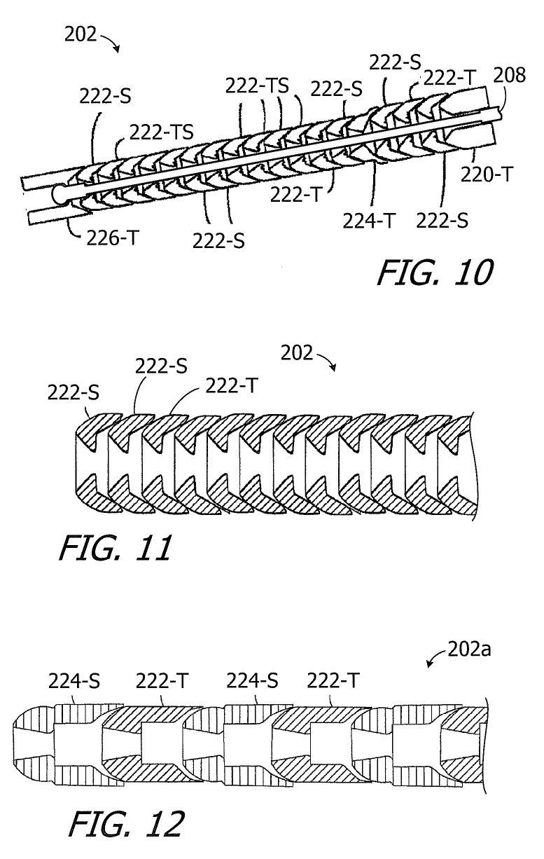

With respect to the other aspects of the exemplary surgical system 10 illustrated in FIG. 1, the flexible articulating arm 200 includes a linkage assembly 202, a bracket 204 that mounts the arm to the supporting structure (e.g. the side rail of an operating table), a tension block 206 that applies tension to the linkage assembly cable 208 (FIG. 10), and a connector 210 that releasably couples the tissue stabilizer apparatus 100 to the arm. The tension block 206 includes a mounting block 212 and a rotatable handle 214. The mounting block 212 may have an internal passage receiving a screw and, affixed to the screw, a transverse pin riding in slots formed in opposite sides of the mounting block. The pin and slots prevents the screw from rotating relative to mounting block 212. The threads of the screw engage internal threads in the rotatable handle 214, which also has an internal shoulder that can engage with the screw's head. The screw is directly attached (or otherwise operably connected to) the cable 208 and, accordingly, the handle 214 may be rotated to selectively increase or decrease the tension on the linkage assembly 202 to fix the orientation of the arm or permit repositioning of the arm. The bracket 204 and mounting block 212 may also be used to fix the location of the flexible articulating arm 200 on the supporting structure. To that end, a screw mechanism 216, including a pivot handle 218, may be used to drive the bracket 204 towards (and away from) mounting block 212.

Turning to FIGS. 10 and 11, the exemplary linkage assembly 202 includes a number of differently shaped links 220, 222, 224 and 226. Each link includes at least one contact surface, which contact couples to a neighboring contact surface of another link. Links 220 and 226 each have exactly one contact surface. The contact surface of link 220 is convex, while the contact surface of link 226 is concave. Links 222 and 224 each have two contact surfaces, one concave and the other convex. At one longitudinal end of the linkage assembly 202, link 226 is coupled with a link 222, while link 220 is coupled with a link 222 at the other longitudinal end. The tension cable 208 extends through the links and is anchored within link 226. An alternative linkage assembly 202a is illustrated in FIGS. 12,13A and 13B and described in greater detail below.

The exemplary links may be formed from various metals and/or combinations thereof and the reference characters associated with each link include a material indicator. More specifically, a “-T” indicates that a link is composed primarily of titanium and a “-S” indicates that a link is composed primarily of stainless steel. With respect to links that employ two or more distinct metallic compounds, e.g. one for each contact surface, a “-TS” indicates that a link has a concave surface primarily composed of a titanium alloy, and a convex surface primarily composed of a stainless steel alloy, while a “-ST” indicates that a link has a concave surface primarily composed of a stainless steel alloy, and a convex surface primarily composed of a titanium alloy.

In the exemplary linkage assembly 202 illustrated in FIGS. 10 and 11, the concave and convex surfaces of the exemplary links 220, 222, 224 and 226 embody shapes, which for their materials, maximize static friction as well as kinetic friction when contacting each other under tension. In some implementations, a first link with a first contact surface (e.g. link 222-T) is composed of a first contact material and a second link with a second contact surface (e.g. link 222-S) is composed of a second contact material, with each of the contact materials primarily composed of a different metallic compound. A high friction coupling between the first link and the second link may created by the first contact surface contacting the second contact surface when induced by the tension cable 208. The first contact surface, composed of the first contact material, contacting the second contact surface, composed of the second contact material, has a higher friction coefficient than results from composing both contact surfaces of either contact material. Suitable friction coefficients may range from, but are not limited to, 0.3 to 0.3875.

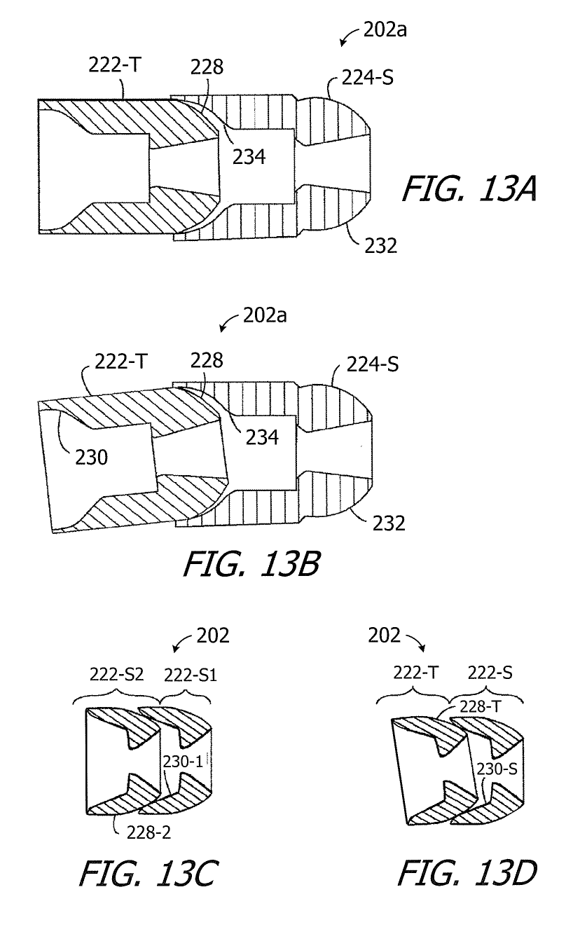

Turning to FIGS. 13A-13B, in the linkage assembly 202a illustrated in FIG. 11, at least two of the links (i.e. links 222-T and 224-S) are coupled through a spherical convex surface contacting a spherical concave surface. The spherical convex surface 228 connects with the semi-spherical concave surface 234. The diameters of the two surfaces are preferably slightly different, with the convex semi-spherical 228 diameter being larger than the semi-spherical diameter of the interfacing concave surface 234. Convex surface 228 and concave surface 234 form an interference fit when the two surfaces contact each other under tension. The wall of link 224-S is sufficiently thin and resilient where the two surfaces come together to provide an area contact between the links.

FIG. 13C shows two stainless steel links (labeled 222-S1 and 222-S2) from the exemplary linkage assembly illustrated in FIG. 10 coupled with a spherical convex surface contacting a conical concave surface. More specifically, the spherical convex surface 228-2 connects with the conical concave surface 230-1. The diameters of the two surfaces are slightly different, with the convex semi-spherical 228-2 diameter being larger than the conical diameter of the interfacing concave surface 230-1. Convex surface 228-2 and concave surface 230-1 form an interference fit when the two surfaces contact each other under tension. The wall of link 222-S1 is sufficiently thin and resilient where the two surfaces come together to provide an area of contact.

In FIG. 13D, links 222-T and 222-S from the exemplary linkage assembly illustrated in FIG. 10 form a coupling where a spherical convex titanium surface contacts a conical concave stainless steel surface, i.e. the spherical convex surface 228-T connects with the conical concave surface 230-S. The diameters of the two surfaces are slightly different, with the convex semi-spherical 228-T diameter being larger than the conical diameter of the interfacing concave surface 230-S. Convex surface 228-T and concave surface 230-S form an interference fit when the two surfaces contact each other under tension. The wall of link 222-S is sufficiently thin and resilient where the two surfaces come together to provide an of area contact.

The circular edge of the opening of each link illustrated in FIGS. 13A-13D may be concentric with the center of the imaginary sphere in which the surface lies when the links are fully engaged with each other. The edge is rounded to avoid a sharp edge that could damage the tensioning cable. The rounded edge has a very small radius of curvature to maximize the contact area of the mating convex and concave surfaces. The fact that the edge is rounded instead of sharp has negligible effect on the contact area.

The diameters of the convex and mating concave link surfaces may vary over the length of the linkage assembly. This supports the need for increased strength and/or stiffness at the proximal end of the articulating arm near the tension block 206, where the applied mechanical moment is greatest. The joints at the proximal end of the arm are preferably larger in diameter. This increases their rotational inertia, or resistance to rotation, in addition to providing greater frictional contact area than smaller distal beads located furthest from tension block 206. The greatest load-bearing link is frequently the most proximal link. This link may be sunk into the body of the articulating column providing a mechanical lock, prohibiting rotation of this link.

One potential mode of failure of a flexible articulating arm that is used repeatedly is cable failure. If the cable fails in an arm with a single uniform cable, nothing is left holding the links together. This allows the links to fall into the surgical field. A variety of factors are associated with the potential for cable failure. The cable (e.g. cable 208) is shortened during use to create compressive forces between adjacent links and rigidify the linkage assembly, which results in tensile fatigue forces being applied to the cable. Shear forces are applied to the strands in contact with the inner radius of the links. If these radii are small, they contact a finite area of the cable and act as a knife edge, greatly wearing a localized area of the cable as it slides over these edges. If the arm is forcefully moved when in the rigid state (when all the slack is already removed from the cable), large loads will stretch the cable strands and greatly accelerate failure.

Various portions of the links may be configured so as to reduce the likelihood of cable failure. For example, the radius of curvature of areas contacting the cable may be increased, as alluded to above. The bend radius of a linkage assembly may be selected based on the minimum radius of curvature permissible for the cable that will be used in conjunction with that linkage assembly. The shape of the adjacent links may be designed to provide a gentle contour creating the selected radius, thereby more evenly distributing the load to more of the cable strands and minimizing contact forces applied to the strands in contact with the links and any sharp edges thereof.

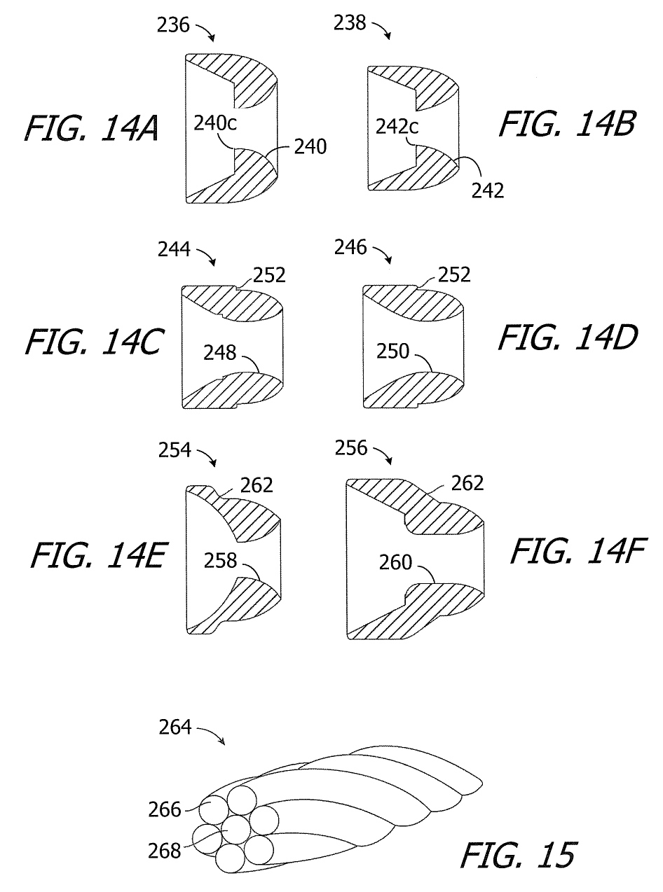

The links illustrated in FIGS. 14A-14F are examples of links that may be employed in the present linkage assemblies to reduce the likelihood of cable failure. Referring first to FIGS. 14A and 14B, links 236 and 238 include inner surfaces 240 and 242 that each have a relatively large radius of curvature. The inner surface corners 240c and 242c may also be rounded in some implementations. The links 244 and 246 illustrated in FIGS. 14C and 14D include inner surfaces 248 and 250 that each have a relatively large radius of curvature. The links 244 and 246 also have an external ridge 252 that prevents the arm assembly from bending beyond a preset limit. The links 254 and 256 illustrated in FIGS. 14E and 14F include inner surfaces 258 and 260 that each have a relatively large radius of curvature. The links 254 and 256 also have an external ridge 262 that prevents the arm assembly from bending beyond a preset limit. The external ridge 262 is more tapered than that illustrated in FIGS. 14C and 14D to provide a smoother external profile of the arm assembly.

Decreasing the coefficient of friction between cable and link contact surfaces also improves the life of the cable. A thin, biocompatible material may be used to provide a hard and lubricious surface. With no surface treatment, the cable may catch on the internal surface of the links causing large contact forces and strains on portions of the cable. The lubricious surface allows the cable to more easily slide along the surfaces of the links as tension is applied, thereby reducing the chance of larger point load or frictional wear on the cable. One option for the lubricious surface is hard chrome plating. The chrome is hard and lubricious, and thus serves as a good material for plating if the desired result is wear resistance. The links, the cable or both may be coated to provide this advantage.

In other implementations, the cable may include a device that will hold the links together despite cable failure. One example of such a cable is generally represented by reference numeral 264 in FIG. 15. The cable 264 includes a plurality of stainless steel strands 266 and at least one elastic (or superelastic) strand 268. When the strands 266 fail, the elastic nature of the strand 268 will cause that portion of the cable 264 to stretch and allow the flexible arm to fail while still holding the links together. One suitable material for the elastic strand 268 is a nickel titanium alloy sold under the trade name Nitinol.

With respect to the manner in which the tissue stabilizer apparatus 100 releasably connected the flexible articulating arm 200 in the illustrated implementation, the exemplary connector 210 (FIG. 1) may be a two-part structure including the outer collar illustrated in FIGS. 16A-16C and the inner cylinder illustrated in FIGS. 17A-17C.

Referring first to FIGS. 17A-17C, the inner cylinder 270 includes a deflectable portion 272, which creates a spring effect, and a spherical surface 274 that is carried by the deflectable portion and is configured to slide along shaft channel 156 and mate with the shaft detent 158 (FIG. 4). Inner cylinder end 276 is secured to the associated arm, and the shaft 148 is inserted at end 278. The collar 280 is movable between a locked position which prevents movement of the shaft 148 and an unlocked position which permits withdrawal of the shaft, and is biased to the locked position by an internal coil spring (not shown). The collar 280 (FIGS. 16A-16C) also includes a necked down portion 282. To insert the tissue stabilizer shaft 148, collar 280 is moved away from cylinder end 276 until the collar 280 is in the unlock position where the neck down portion 282 does not apply force to the deflectable portion 272. After the shaft 148 is inserted and the spherical surface 274 of the deflectable portion 272 mates with the spherical concave detent 226, the collar 280 may be released. The spring (not shown) forces the collar 280 back to the lock position, where the neck down portion 282 comes into contact with the deflectable portion 272, forcing the spherical surface 274 to seat in the shaft detent 158, and locking the axial and rotational position of the tissue stabilizer apparatus. Suitable materials for the inner cylinder 270 and collar 280 include stainless steel.

Additional details concerning the exemplary flexible articulating arms described above, as well as other arms, are provided in U.S. Pat. No. 6,860,668 and U.S. Patent Pub. No. 2005/0226682 A1, which are incorporated herein by reference.

Although the inventions disclosed herein have been described in terms of the preferred embodiments above, numerous modifications and/or additions to the above-described preferred embodiments would be readily apparent to one skilled in the art. It is intended that the scope of the present inventions extend to all such modifications and/or additions and that the scope of the present inventions is limited solely by the claims set forth below.

1. A tissue stabilizer apparatus, comprising:
a tissue stabilizer including
a frame having a resilient portion, and
at least one suction member having at least one suction port carried by the frame; and
a connector associated with the frame and configured to secure the tissue stabilizer to a mechanical arm.

2. A tissue stabilizer apparatus as claimed in claim 1, further comprising:
a frame port operably connected to the at least one suction port.

3. A tissue stabilizer apparatus as claimed in claim 2, wherein the frame includes an interior lumen that is connected to the frame port and at least one aperture that is connected to the interior lumen and exposed to the at least one suction port.

4. A tissue stabilizer apparatus as claimed in claim 3, wherein
the frame includes a plurality of apertures; and
the suction member includes a plurality of suction ports that are respectively exposed to the plurality of apertures.

5. A tissue stabilizer apparatus as claimed in claim 1, wherein
the frame supports first and second suction members and the suction members are separated from one another by a gap; and
the frame is configured such that it can be deflected to a point at which the gap has been reduced by at least 25% without substantial plastic deformation of the frame.

6. A tissue stabilizer apparatus as claimed in claim 5, wherein
the suction members define distal regions; and
the frame is configured such that it can be deflected to a point at which the distal regions of the suction members contact one another without substantial plastic deformation of the frame.

7. A tissue stabilizer apparatus as claimed in claim 1, wherein
the frame comprises a substantially U-shaped frame including a curved portion and a pair of substantially straight portions; and
the at least one suction member comprises a pair of suction members respectively carried by the pair of substantially straight portions of the frame.

8. A tissue stabilizer apparatus as claimed in claim 7, wherein
the substantially straight portions define respective proximal ends and distal ends; and
the distance between the distal ends of the substantially straight portions is greater than the distance between the proximal ends of the substantially straight portions.

9. A tissue stabilizer apparatus as claimed in claim 1, wherein the connector is configured to releasably secure the tissue stabilizer to the mechanical arm.

10. A tissue stabilizer apparatus as claimed in claim 9, wherein the connector includes a shaft with a spherical indentation.

11. A tissue stabilizer apparatus, comprising:
a tissue stabilizer, including first and second suction zones and defining an initial shape, configured such that at least one of the first and second suction zones will move a distance at least 1 mm in response to the application of a force of at least one pound thereto and the tissue stabilizer will return to the initial shape when the force is removed; and
a connector associated with the frame and configured to secure the tissue stabilizer to a mechanical arm.

12. A tissue stabilizer apparatus as claimed in claim 11, wherein the tissue stabilizer is configured such that both of the first and second suction zones will move a distance at least 1 mm when respective forces of at least one pound are applied thereto and the tissue stabilizer will return to the initial shape when the forces are removed.

13. A tissue stabilizer apparatus as claimed in claim 12, wherein the tissue stabilizer includes first and second fulcrums about which the first and second suction zones deflect in response to the application of respective forces thereto.

14. A tissue stabilizer apparatus as claimed in claim 11, wherein each suction zone includes a plurality of suction ports.

15. A tissue stabilizer apparatus as claimed in claim 11, wherein the tissue stabilizer includes a fulcrum about which the at least one of the first and second suction zones deflects in response to the application of a force thereto.

16. A tissue stabilizer apparatus as claimed in claim 11, wherein the tissue stabilizer is substantially U-shaped.

17. A tissue stabilizer apparatus as claimed in claim 16, wherein
the substantially U-shaped tissue stabilizer include a curved portion and a pair of substantially straight portions; and
the first and second suction zones are associated with the substantially straight portions.

18. A tissue stabilizer apparatus as claimed in claim 17, wherein
the substantially straight portions define respective proximal ends and distal ends; and
the distance between the distal ends of the substantially straight portions is greater than the distance between the proximal ends of the substantially straight portions.

19. A tissue stabilizer apparatus as claimed in claim 11, wherein the connector is configured to releasably secure the tissue stabilizer to the mechanical arm.

20. A tissue stabilizer apparatus as claimed in claim 19, wherein the connector includes a shaft with a spherical indentation.

21. A surgical system, comprising:
an arm; and
a tissue stabilizer, operably connected to the arm, including a frame having a resilient portion and at least one suction member having at least one suction port carried by the frame.

22. A surgical system as claimed in claim 21, wherein the arm comprises a flexible articulating arm.

23. A surgical system as claimed in claim 22, wherein the flexible articulating arm includes a plurality of links and a tension cable.

24. A surgical system as claimed in claim 21, wherein
the arm includes a first connector;
the tissue stabilizer includes a second connector; and
the first and second connectors are configured to releasably connect the tissue stabilizer to the arm.

25. A surgical system as claimed in claim 21, further comprising:
a frame port operably connected to the at least one suction port.

26. A surgical system as claimed in claim 25, wherein the frame includes an interior lumen that is connected to the frame port and at least one aperture that is connected to the interior lumen and exposed to the at least one suction port.

27. A surgical system as claimed in claim 26, wherein
the frame includes a plurality of apertures; and
the suction member includes a plurality of suction ports that are respectively exposed to the plurality of apertures.

28. A surgical system as claimed in claim 21, wherein
the frame supports first and second suction members and the suction members are separated from one another by a gap; and
the frame is configured such that it can be deflected to a point at which the gap has been reduced by at least 25% without substantial plastic deformation of the frame.

29. A surgical system as claimed in claim 28, wherein
the suction members define distal regions; and
the frame is configured such that it can be deflected to a point at which the distal regions of the suction members contact one another without substantial plastic deformation of the frame.

30. A surgical system as claimed in claim 21, wherein
the frame comprises a substantially U-shaped frame including a curved portion and a pair of substantially straight portions; and
the at least one suction member comprises a pair of suction members respectively carried by the pair of substantially straight portions of the frame.

31. A surgical system as claimed in claim 30, wherein
the substantially straight portions define respective proximal ends and distal ends; and
the distance between the distal ends of the substantially straight portions is greater than the distance between the proximal ends of the substantially straight portions.

32. A surgical system, comprising:
an arm; and
a tissue stabilizer, operably connected to the arm, that includes first and second suction zones, defines an initial shape, and is configured such that at least one of the first and second suction zones will move a distance at least 1 mm in response to the application of a force of at least one pound thereto and the tissue stabilizer will return to the initial shape when the force is removed.

33. A surgical system as claimed in claim 32, wherein the arm comprises a flexible articulating arm.

34. A surgical system as claimed in claim 33, wherein the flexible articulating arm includes a plurality of links and a tension cable.

35. A surgical system as claimed in claim 32, wherein
the arm includes a first connector;
the tissue stabilizer includes a second connector; and
the first and second connectors are configured to releasably connect the tissue stabilizer to the arm.

36. A surgical system as claimed in claim 32, wherein the tissue stabilizer is configured such that both of the first and second suction zones will move a distance at least 1 mm when respective forces of at least one pound are applied thereto and the tissue stabilizer will return to the initial shape when the forces are removed.

37. A surgical system as claimed in claim 36, wherein the tissue stabilizer includes first and second fulcrums about which the first and second suction zones deflect in response to the application of respective forces thereto.

38. A surgical system as claimed in claim 32, wherein each suction zone includes a plurality of suction ports.

39. A surgical system as claimed in claim 32, wherein the tissue stabilizer includes a fulcrum about which the at least one of the first and second suction zones deflects in response to the application of a force thereto.

40. A surgical system as claimed in claim 32, wherein the tissue stabilizer is substantially U-shaped.

41. A surgical system as claimed in claim 40, wherein
the substantially U-shaped tissue stabilizer include a curved portion and a pair of substantially straight portions; and
the first and second suction zones are associated with the substantially straight portions.

42. A surgical system as claimed in claim 41, wherein
the substantially straight portions define respective proximal ends and distal ends; and
the distance between the distal ends of the substantially straight portions is greater than the distance between the proximal ends of the substantially straight portions.
